(19) United States
(12) Patent Application Publication
(10) Pub. No.: US 2019/0321205 A1
(43) Pub. Date: Oct. 24, 2019

(54) HANDLE WHEEL IMPLANT DELIVERY DEVICE

(71) Applicant: Q3 MEDICAL DEVICES LIMITED, Dublin (IE)

(72) Inventors: Martina Schmitt, Winsen (Luhe) (DE); Eric K. Mangiardi, Charlotte, NC (US)

(21) Appl. No.: 15/956,905

(22) Filed: Apr. 19, 2018

(51) Int. Cl. A61F 2/95

(52) U.S. Cl. CPC A61F 2/95; A61F 2002/9517

(57) ABSTRACT

A medical implant delivery device for deploying an intraluminal medical device in a body lumen is disclosed. Also disclosed is a method for deploying an intraluminal medical device using the medical implant delivery device and a kit comprising the medical implant delivery device.

### FIELD

The present application relates generally to medical devices and, in particular, to a handle wheel implant delivery device for introducing implantable medical devices into a body cavity.

### BACKGROUND

Implants may be placed in the human body for a variety of reasons. For example, stents are placed in a number of different body lumens such as blood vessels and biliary ducts; vena cava filters are implanted in the vena cava to catch thrombus sloughed off from other sites within the body; and vaso-occlusive devices are used for the treatment of intravascular aneurysms.

Interventional practitioners, regardless of subspecialty have always had to demonstrate profound dexterity in order to effectively and accurately perform invasive procedures. This is particularly the case with the delivery and deployment of implantable devices where there is very little room for error with respect to placement. In order to assist with placement accuracy, many interventionalists utilize scopes, such as bronchoscopes or endoscopes, ultrasound, ct scanning, or other imaging modalities. However, handling the imaging modality and the delivery catheter can often be a clumsy process when the two devices easily disassociate from each other. Moreover, since many delivery catheters, for one reason or another, cannot be adequately managed with one hand, additional personnel are required when handling the scope and the delivery catheter.

Therefore, there is an existing need for a delivery system that allows a physician to deploy an implantable device with one hand.

### SUMMARY

One aspect of the present invention relates to a medical implant delivery device for deploying an intraluminal medical device in a body lumen includes a housing comprising a handle, the housing comprising a distal housing end and a proximal housing end, the distal housing end being configured for attachment to a deployment catheter carrying the intraluminal support device; a finger-actuated trigger connected to an engagement bar that is interfaced with a first rotating gear mounted on a first axle connected to the housing; a second rotating gear mounted on a second axle connected to the housing, wherein the second rotating gear is interfaced with the first rotating gear; a cam mounted to the second axle in a concentric relationship with the second rotating gear, the cam having a larger diameter than the second rotating gear; and a tether attached to the cam, wherein the trigger is laterally displaceable such that movement of the trigger in a proximal direction rotates the first gear in a counterclockwise direction, and causes the second gear to rotate in a clockwise direction, and wherein the clockwise rotation of the second gear causes the cam to pull the tether in a proximal direction during actuation of the trigger so that when the tether is operably linked to the outer sheath of a deployment catheter the tether causes the outer sheath to retract in a proximal direction, thereby releasing the expandable intraluminal support device in a body lumen.

In one embodiment, the method comprises the steps of repeatedly pulling and releasing the trigger such that the engagement bar disengages from the first gear at a first location and then reengages a more distal portion of the engagement bar at a second location for further retraction of the outer sheath each time the trigger is actuated.

In another embodiment, the method comprises the step of turning a cam in the form of a rotatable thumbwheel containing grippable structures on an outer periphery in a clockwise manner to provide finer manual adjustments to outer sheath retraction relative to the trigger.

In certain embodiments, the delivery device includes one or more locking structures for preventing inadvertent displacement of the outer sheath.

In one embodiment, the cam is entirely enclosed within the housing.

In another embodiment, a portion of the cam extends beyond the surface of the housing, such that the cam is a rotatable thumbwheel comprising grippable structures on an outer periphery facilitating finer manual adjustments to outer sheath retraction relative to the trigger.

In a particular embodiment, the delivery device is further configured so that upon pulling and releasing the trigger, the engagement bar disengages from the first gear at a first location and then reengages a more distal portion of the engagement bar at a second location for further displacement of the outer sheath each time the trigger is actuated.

Another aspect of the present application relates to a method for delivering an implantable intraluminal medical device to a body lumen of a subject. The method includes the steps of: holding a delivery device of the present application, wherein the delivery device is operably linked to a deployment catheter comprising an outer sheath covering an intraluminal medical device crimped over an inner sheath comprising an inner sheath lumen configured for passing a guide wire therethrough; running a guide wire through the inner sheath lumen of the deployment catheter and advancing the guide wire through a body lumen of a subject to a site for deployment of the medical device; advancing the catheter over the guide wire until the outer sheath extends over the site for deployment of the medical device; actuating the trigger of the delivery device, thereby retracting the outer sheath in the delivery device, releasing the intraluminal device from the catheter into the body lumen, or both.

In one embodiment, the trigger is repeatedly pulled and released such that the engagement bar disengages from the first gear at a first location and then reengages a more distal portion of the engagement bar at a second location for further displacement of the outer sheath each time the trigger is actuated.

In another embodiment, the method comprises the use of a cam in the form of a rotatable thumbwheel containing grippable structures on an outer periphery that is turned in a clockwise manner to provide finer manual adjustments to outer sheath retraction relative to the trigger.

Another aspect of the present application relates to a kit for deploying an intraluminal medical device. The kit comprises the medical implant delivery device of the present application and instructions on how to use the device.

Further objectives, features and advantages of the invention will be apparent from the following detailed description taken in conjunction with the accompanying drawings.

### BRIEF DESCRIPTION OF THE DRAWINGS

For the purposes of this disclosure, unless otherwise indicated, identical reference numerals used in different figures refer to the same component.

FIG. 1 shows a view of an embodiment of an exemplary delivery system connected to a deployment catheter by a connector.

FIG. 2 shows a view of an exemplary embodiment of a trigger system of the delivery system.

### DETAILED DESCRIPTION

While this invention may be embodied in many different forms, there are described in detail herein specific preferred embodiments of the invention. This description is an exemplification of the principles of the invention and is not intended to limit the invention to the particular embodiments illustrated.

Unless defined otherwise, all technical and scientific terms used herein have the same meanings as commonly understood by one of skill in the art to which the disclosed method and compositions belong. It must be noted that as used herein and in the appended claims, the singular forms “a,” “an,” and “the” include plural reference unless the context clearly dictates otherwise. With respect to documents described in the present application, all of the teachings in any issued patent or patent application publication described in this application is expressly incorporated by reference herein.

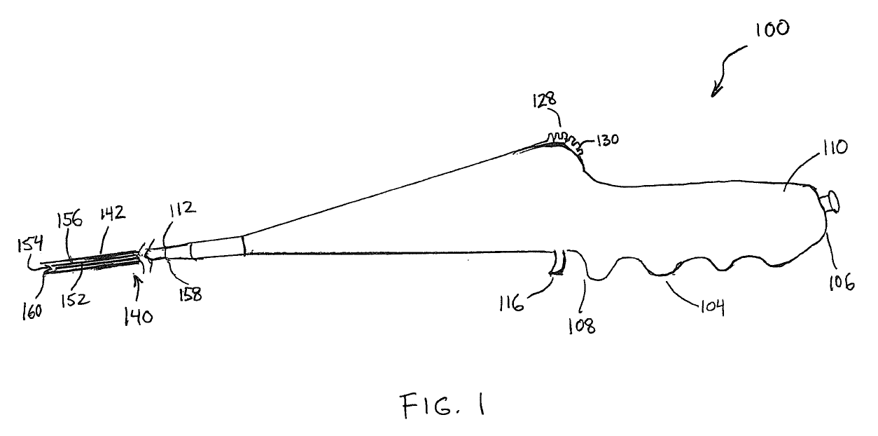

One aspect of the present invention relates to an intraluminal implant delivery device 100 for deploying an implantable medical device in a body lumen. As shown in FIG. 1, the delivery device 100 comprises a housing 110 having a handle 104, the handle 104 comprising a proximal end 106 and a distal end 108. The housing 110 includes a proximal end 106 and a distal housing end 112 connectable to the proximal end of a deployment catheter 140 carrying the intraluminal support device 142. The deployment catheter 140 comprises a distal end 144 and a proximal end 146 connecting to the distal end 112 of the delivery device 100. The deployment catheter 140 includes an inner sheath 152 comprising a longitudinal lumen 154 through which a guide wire can be fed to facilitate delivery of the expandable intraluminal support device 142. The deployment catheter 140 further includes an outer sheath 156 comprising a longitudinal lumen 160 in which the intraluminal device 142 is disposed. In particular, the expandable intraluminal support device 142 is compressed around the inner sheath 152 near the distal end 144 of the deployment catheter 140, whereby the outer sheath 156 covers the expandable intraluminal support device 142.

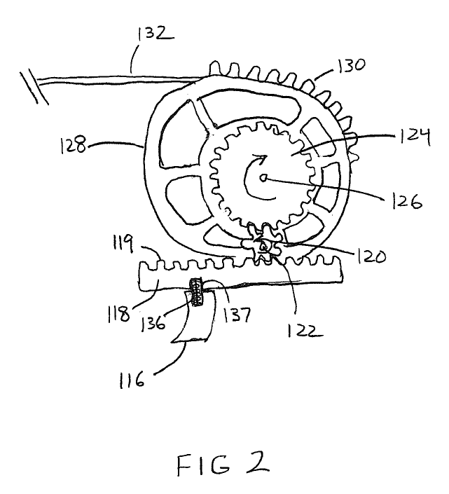

As shown in FIG. 2, the handle 104 includes structural components for retracting the outer sheath 156 of the deployment catheter 140 to release the expandable intraluminal support device 142 when connectively linked thereto. In particular, the handle 104 is proximal to a finger-actuated trigger 116 connected to an gear engagement bar 118 interfaced by way of a plurality of engagement members 119 with a first rotating gear 120 mounted on a first axle 122 connected to the housing 110. The first rotating gear 120 is further interfaced with a second rotating gear 124 mounted on a second axle 126 connected to the housing 110. In some embodiments, the second rotating gear 124 has a diameter that is larger than that of the first rotating gear 120. In some embodiments, the second rotating gear 124 has a diameter that is smaller than that of the first rotating gear 120. The second axle 126 concentrically joins the second rotating gear 124 and a cam 128. The cam 128 is attached to a proximal end 134 of a tether 130 and has a larger diameter than the diameter of the second gear 124. The tether 130 is connectable to the outer sheath 156 of the deployment catheter 140 at the proximal end 158 of the outer sheath 156.

Preferably, the cam 128 has a diameter exceeding the combined diameters of the first and second gears 120, 124. In one embodiment, the cam 128 is enclosed in the housing 110. In another embodiment, a portion of the cam 128 extends beyond the surface of the delivery housing or handle 104 to allow finer adjustment control of outer sheath 156 retraction relative to the level of outer sheath 156 retraction effected when actuating the trigger 116. In this embodiment, the cam 128 further includes finger grippable (e.g., toothlike) structures 132 or knurling on the outer periphery forming a rotatable thumbwheel that can be engaged by the practitioner for direct retraction of the tether 130 and the outer sheath 156.

As shown in FIG. 2, the trigger 116 is laterally displaceable, whereby movement of the trigger 116 in a proximal direction rotates the first gear 120 in a counterclockwise direction through the interaction between the engagement members 119 in the engagement bar 118 and the first gear 120. Rotation of the first gear 120 causes the second gear 124 to rotate in a clockwise direction. Clockwise rotation of the second gear 124 causes the cam 128 to pull the tether 130 in a proximal direction during actuation of the trigger 116. When the delivery device 100 is connected to the deployment catheter 140, more specifically when the tether 130 is operably linked to the outer sheath 156, pulling on the tether 130 causes the outer sheath 156 to retract in a proximal direction, thereby exposing and positioning the expandable intraluminal support device 142 in a body lumen. More particularly, a small lateral displacement of the trigger 116 translates into a larger lateral displacement of the tether 130 and by extension, a larger lateral displacement of the outer sheath 156.

In some embodiments, the delivery device 100 allows for both clockwise and counterclockwise rotation of the cam 128. A practitioner can push the trigger 116 in a distal direction, thereby reversing the above-described gear rotations to displace the outer sheath 156 in a distal direction and/or achieve the original starting configuration of the trigger 116 in the delivery device 100.

In some embodiments, the delivery device 100 allows for only clockwise rotation of the cam 128 to prevent back movement of the outer sheath 156 when the trigger 116 is released from finger pressure. In some embodiments, the first gear 120 can only rotate counterclockwise.

In some embodiments, the first gear 120 is part of a first gear assembly 123 (not shown in the preliminary drawings) consisting of several first gears 120 of varying diameters. The user of the delivery device 100 may change the degree to which the outer sheath 156 can be laterally displaced by selectively engaging the second gear 124 with any one of the individual first gears 120 in the first gear assembly 123. When rotatably engaging a larger first gear 120 in the first gear assembly 123, a smaller lateral displacement of the outer sheath 156 for each trigger pull can be achieved. Alternatively, or in addition, the second gear 124 may be part of a second gear assembly 127 (not shown in the preliminary drawings) consisting of one or more second gears 124 of varying diameters in which the first gear 120 in a first assembly 123 or otherwise selectively engages any one of the individual second gears 124 in the second gear assembly 127. Consequently, when rotatably engaging a larger second gear 124 in the second gear assembly 127, a smaller lateral displacement of the outer sheath 156 for each trigger pull can be achieved.

In some embodiments, the device 100 is structurally configured so that actuation of the trigger 116 in a proximal direction displaces the engagement bar 118 a predetermined distance in the proximal direction from a first position to a second position so that upon release of the trigger 116, the engagement bar 118 disengages from the first gear 120 at the second position and then reengages a more proximal portion of the first gear 120 for further lateral displacement of the engagement bar 118 and, by extension, the outer sheath 156 in a ratcheting manner analogous to a socket wrench.

In one embodiment, the trigger 116 is connected to the engagement bar 118 through a spring-loaded pin 136 on the trigger 116 configured for mated attachment to one or more holes 137 in the trigger side of the engagement bar 118. In another embodiment, the trigger 116 is connected to the engagement bar 118 through a spring-loaded pin 136 on the engagement bar 118 configured for mated attachment to one or more holes 137 in the trigger 116. In both embodiments, the pin-and-hole connection between the trigger 116 and the engagement bar 118 allows for the engagement bar 118 to be lowered in the direction of the trigger 116 so that the first gear 120 disengages from the engagement bar 118 at the end of each trigger pull. In these embodiments, the engagement bar 118 is initially engaged with the first gear 120 at a starting position before the trigger pull. After a full trigger pull, the engagement bar 118 is laterally displaced until it reaches a designated disengagement position such that the engagement bar 118 disengages from the first gear 120 upon release of the trigger 116, and then re-engages the first gear 120 at the original starting position before the trigger pull via the spring loaded pin 136 and hole(s) 137.

The engagement bar 118, gears 120, 124, cam 128 and engagement members 119 can vary in size and distribution, both within a given gear assembly or independent of whether one or more gear assemblies are included. In some embodiments, the length of the engagement bar 118 ranges between 50 to 200 mm and the engagement members 119 are separated from one another by a distance between 0.2 and 2 mm. In some embodiments, the gear 120, 124 diameters range between may range between 2 mm and 20 mm. The cam 128 diameter may range between 5 and 50 mm.

The individual components in the delivery device 100 may be made from any medically suitable materials, including metallic and polymeric materials. The choice of materials may vary depending on e.g., whether the device is re-usable or disposable. For example, where the delivery device 100 is re-usable, it must be sterilized before use (by e.g., autoclaving). Accordingly, a re-usable delivery 100 must include materials (e.g., metallic materials) that can maintain their integrity following autoclaving, whereas a disposable delivery device 100 can be made from polymeric materials.

In one embodiment, the deployment catheter 140 is integrally linked to the delivery device 100. In this embodiment, the tether 130 directly links the cam 128 to the outer sheath 156 of the deployment catheter 140.

In another embodiment, the deployment catheter 140 and the delivery device are separate, stand-alone components, whereby the tether 130 is operably linked to the outer sheath 156 of the deployment catheter 140. In this case, the tether 130 may be operably linked to the outer sheath 156 by any suitable attachment means, including but not limited to the use of hooks, pins, or extensions in one component with complementary joining structures in the other component.

The distal end of the delivery device 100 may be configured to accommodate or allow for the attachment of variable catheter shafts from a variety of deployment catheters for ease of manufacturing and interchangeability of varying catheter diameters. In one embodiment, the distal handle end 108 or distal housing end 112 of the delivery device 100 is configured so that a deployment catheter 140 can be removably attached to one of the distal ends 108, 112 of the delivery device 100. For example, the deployment catheter 140 may be screwed onto a distal end 108, 112, or coupled to the device 100 by other conventional means such as a luer, hub, or other standard attachment mechanism. In some embodiments, a Y-shaped connector may be used to connectively link the delivery device 100 to the deployment catheter 140. The connector may be structurally configured to operably join these components and/or provide a means for entry and delivery of materials (e.g., guide wires, fluids) through the deployment catheter 140. The connector can allow for the attachment of a fluid reservoir or syringe. In particular, the fluid reservoir or syringe may comprise an opacity enhancing substance to allow visualization of the balloon when it is inflated.

In certain embodiments, the device 100 may include or may be embedded with one or more marker elements or contrast agents to allow for visualization of the intraluminal device 142, outer sheath 156, inner sheath 152, and/or inflation balloon during delivery to insure proper placement and visibility during the deployment using fluoroscopy, x-ray, or other imaging modalities. The marker elements may include metallic particles, opacifying substances and combinations thereof.

As used herein, the terms “opacifying substance”, “radio-opaque substance” and “radio-opaque marker” refers to a medical contrast medium or contrast agent that increases or enhances the visibility of the intraluminal device 142 or other component of the deployment catheter 140 within the body of a subject for imaging device components during a device deployment. Opacifying or radio-opaque substances may be used in particulate, liquid, powder or other suitable form as part of any device component or as part of a perfusion medium or inflation medium. Exemplary opacity enhancing substances include, but are not limited to: iodine compounds, barium compounds, such as barium sulfate and barium iridium, fluorescent dyes, microbubbles, metallic materials, such as iron, tungsten, gold, platinum, gadolinium, bismuth and tantalum. Iodine compounds can be ionic (high osmolar) or non-ionic (low osmolar) compounds. Exemplary ionic iodine compounds may comprise diatrizoic acid, metrizoic acid, ioglicic acid, or salts thereof. Exemplary non-ionic iodine compounds may comprise iopadimol, iohexol, ioxilan, iopromide, and iodixanol. Microbubbles are bubbles composed of nitrogen or perfluorocarbon gas smaller than one millimeter in diameter that may be encapsulated with a solid shell, made from a polymer, lipid or a protein such as serum albumin.

Exemplary types of intraluminal medical devices 142 suitable for use with the present delivery system include self-expanding stents, balloon-expandable stents, prosthetic valves, filters, occluders, grafts, stent-grafts, filters, plugs, valve devices, occluders, expandable filters, distal protection devices, and the like. The delivery devices 100 described herein are believed to be particularly well-suited for use with expandable intraluminal medical devices. As used herein, the term “graft” refers to natural and synthetic sections of material, such as polymeric materials, natural tissue, and other flexible sections of material.

In certain embodiments, the device 100 includes one or more locking members to prevent inadvertent displacement of the outer sheath 156.

The dimensions of the deployment catheter 140 must offer enough space for an implantable intraluminal device 142, such as an expandable stent crimped on the inner sheath of the deployment catheter 140. Preferably, the individual parts of the delivery device 100 have smooth outer and inner surfaces to provide low friction between moving parts.

The delivery device 100 of the present application allows the user to install the intraluminal medical device 142 with one hand. The delivery device 100 may be structurally shaped in any ergonomically suitable manner to facilitate gripping and adjustable control of components for retracting the outer sheath and/or other components for device placement and delivery.

The delivery device 100 may be made of any biocompatible material with suitable hardness and rigidity for the delivery of the implantable medical device. The delivery device 100 may be made from plastic materials, preferably moldable plastic materials, in combination with one or more metallic materials. For example, the inner and outer sheaths may be made from a polymer or other suitable material. Exemplary polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), polyether block amides that are free of plasticizers, ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, mixtures, combinations, copolymers thereof, polymer/metal composites, and the like.

The deployment catheter 140 should have sufficient flexibility to adapt to anatomical curvatures without loss of ability to push or pull. Exemplary deployment catheters 140 for use with the delivery device 100 are described in U.S. Pat. No. 9,414,943.

The tether 130 may be one long piece of unitary material or a plurality of pieces, including braided and/or multifilament textile materials. As such, the tether 130 may be made from any suitable material, including metallic materials, such as wires; monofilament or multifilament textile yarns or fibers; suture materials, such as DACRON™, polyester materials, and the like.

In another aspect, the present application provides a method for deploying an intraluminal medical device medical in an anatomical lumen of a patient. In one embodiment, a method for delivering an implantable intraluminal medical device 142 using the delivery device 100 of the present application includes the steps of: operably linking the outer sheath 156 of a deployment catheter 140, the deployment catheter 140 comprising an intraluminal medical device 142 in a pre-deployment configuration, to the cam 128 of the delivery device 100; passing the deployment catheter 140 over a guide wire inserted in a subject's vasculature and advancing the deployment catheter 140 so that the intraluminal medical device 142 is positioned at a desired placement site in a body lumen; and laterally displacing the trigger 116 of the delivery device 100 in a proximal direction, thereby retracting the outer sheath 156, releasing the intraluminal device in the body lumen, or both.

In one embodiment, the method comprises the steps of repeatedly pulling and releasing the trigger 116 such that the engagement bar 118 disengages from the first gear 120 at a first location and then reengages a more distal portion of the engagement bar 118 at a second location for further retraction of the outer sheath each time the trigger 116 is actuated.

In another embodiment, the method comprises the step of turning a cam 128 in the form of a rotatable thumbwheel containing grippable structures 130 on an outer periphery in a clockwise manner to provide finer manual adjustments to outer sheath 156 retraction relative to the trigger 116.

Using the medical implant delivery device of the present application, a variety of target tissues can be imaged, diagnosed, treated, and evaluated with the devices, methods, and systems described herein. In particular, the devices are useful for treating tissues that are accessible via the various lumens of the body, including, but not limited to, blood vessels, vasculature of the lymphatic and nervous systems, structures of the gastrointestinal tract (lumens of the small intestine, large intestine, stomach, esophagus, colon, pancreatic duct, bile duct, hepatic duct), lumens of the reproductive tract (vas deferens, uterus and fallopian tubes), structures of the urinary tract (urinary collecting ducts, renal tubules, ureter, and bladder), and structures of the head and neck and pulmonary system (sinuses, parotid, trachea, bronchi, and lungs). Accordingly, the devices, methods, and systems of the invention may be beneficial in the treatment of a number of disorders, including, but not limited to, atherosclerosis, ischemia, coronary blockages, thrombi, occlusions, stenosis, aneurysms, and the like.

Another aspect of the present application relates to a kit for deploying an intraluminal medical device. The kit comprises the medical implant delivery device of the present application and instructions on how to use the device 100. In some embodiments, the kit further comprises one or more intraluminal medical devices, such as stents, valves and filters. In some embodiments, the kit further comprises one or more self-expandable stents. In some embodiments, the kit further comprises one or more catheters. In some embodiments, the kit further comprises an endoscope.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A medical implant delivery device for deploying an intraluminal medical device in a body lumen, the device comprising:
a housing comprising a handle, the housing comprising a distal housing end and a proximal housing end, the distal housing end being configured for attachment to a deployment catheter carrying the intraluminal support device;
a finger-actuated trigger connected to an engagement bar that is interfaced with a first rotating gear mounted on a first axle connected to the housing;
a second rotating gear mounted on a second axle connected to the housing, wherein the second rotating gear is interfaced with the first rotating gear;
a cam mounted to the second axle in a concentric relationship with the second rotating gear, the cam having a larger diameter than the second rotating gear; and
a tether attached to the cam,
wherein the trigger is laterally displaceable such that movement of the trigger in a proximal direction rotates the first gear in a counterclockwise direction, and causes the second gear to rotate in a clockwise direction, and
wherein the clockwise rotation of the second gear causes the cam to pull the tether in a proximal direction during actuation of the trigger so that when the tether is operably linked to the outer sheath of a deployment catheter the tether causes the outer sheath to retract in a proximal direction, thereby releasing the expandable intraluminal support device in a body lumen.

2. The delivery device of claim 1, wherein the device is integrally linked to a deployment catheter comprising an outer sheath covering an intraluminal medical device crimped over an inner sheath comprising a inner sheath lumen configured for passing a guide wire therethrough, and wherein the tether directly links the cam to the outer sheath.

3. The delivery device of claim 1, wherein the medical device is an expandable stent.

4. The delivery device of claim 1, wherein the distal end of the device is structurally configured so that it can be operably connected to a deployment catheter comprising an outer sheath covering an intraluminal medical device crimped over an inner sheath comprising an inner sheath lumen configured for passing a guide wire therethrough.

5. The delivery device of claim 1, further comprising one or more locking structures for preventing inadvertent displacement of the outer sheath.

6. The delivery device of claim 1, wherein the cam is enclosed within the housing.

7. The delivery device of claim 1, wherein a portion of the cam extends beyond the surface of the housing, and wherein the cam is a rotatable thumbwheel comprising grippable structures on an outer periphery facilitating finer manual adjustments to outer sheath retraction relative to the trigger.

8. The delivery device of claim 1, wherein the device is further configured so that upon pulling and releasing the trigger, the engagement bar disengages from the first gear at a first location and then reengages a more distal portion of the engagement bar at a second location for further displacement of the outer sheath each time the trigger is actuated.

9. A method for delivering an implantable intraluminal medical device to a body lumen of a subject, comprising the steps of:
holding the delivery device of claim 1, wherein the delivery device is operably linked to a deployment catheter comprising an outer sheath covering an intraluminal medical device crimped over an inner sheath comprising an inner sheath lumen configured for passing a guide wire therethrough;
running a guide wire through the inner sheath lumen of the deployment catheter and advancing the guide wire through a body lumen of a subject to a site for deployment of the medical device;
advancing the catheter over the guide wire until the outer sheath extends over the site for deployment of the medical device;
actuating the trigger of the delivery device, thereby retracting the outer sheath in the delivery device, releasing the intraluminal device from the catheter into the body lumen, or both.

10. The method of claim 9, wherein the delivery device integrally linked to the deployment catheter, and wherein the tether directly links the cam in the delivery device to the outer sheath in the deployment catheter.

11. The method of claim 9, wherein a deployment catheter is operably connected to the delivery device.

12. The method of claim 9, wherein the trigger is pulled and released, wherein upon release of the trigger, the engagement bar disengages from the first gear at a first location and then reengages a more distal portion of the engagement bar at a second location to facilitate further displacement of the outer sheath each time the trigger is actuated.

13. The method of claim 9, wherein the cam is a rotatable thumbwheel comprising grippable structures on an outer periphery extending beyond the surface of the housing, and wherein the grippable structures on the outer periphery are displaced in a clockwise manner thereby retracting the outer sheath in the delivery catheter, releasing the intraluminal device from the catheter into the body lumen, or both.

14. The method of claim 9, wherein the implantable medical device is an expandable stent.

15. The method of claim 9, wherein the body lumen is a blood vessel or a bile duct.
